(19) United States
(12) Patent Application Publication
(10) Pub. No.: US 2016/0346190 A1
(43) Pub. Date: Dec. 1, 2016

(54) LATEX OF SPHERICAL PARTICLES WITH IMPROVED THERMAL AGEING PROPERTIES

(71) Applicant: ARKEMA FRANCE, Colombes (FR)

(72) Inventor: Rabi INOUBLI, PAU (FR)

(73) Assignee: ARKEMA FRANCE, Colombes (FR)

(21) Appl. No.: 15/112,881

(22) PCT Filed: Jan. 20, 2015
(86) PCT No.: PCT/FR2015/050134

§ 371 Date: Jul. 20, 2016

(30) Foreign Application Priority Data
Jan. 21, 2014 (FR) 1450444

(51) Int. Cl. A61K 8/90; A61K 8/02; B82Y 40/00; C09K 8/536; C08F 293/00; C09D 153/00; C09K 8/52; A61Q 19/00; B82Y 30/00

(52) U.S. Cl. CPC A61K 8/90; A61Q 19/00; A61K 8/025; B82Y 40/00; B82Y 30/00; C08F 293/005; C09D 153/00; C09K 8/52; C09K 8/536; A61K 2800/10; A61K 2800/413

(57) ABSTRACT

The present invention relates to the use of polymer particles in the form of spherical micelles constituted of block copolymers, as additives for aqueous or organic solutions. More particularly, the invention relates to the use of said spherical polymer particles for improving the ageing resistance of an aqueous or organic composition.

The present invention relates to polymer particles with a spherical shape constituted of block copolymers, and to the thermal ageing resistance properties thereof.

The synthesis of a latex of nanometric-sized spherical (meth)acrylic or other particles is known.

The publication by G. Delaittre et al. Chem. Commun. 2009, 2887-2889 describes the preparation of copolymer vesicles containing PNaA-b-P4VP amphiphilic blocks. These copolymers are obtained in aqueous medium, at pH 11, in a single step, by emulsion CRP; the polymerization reaction of 4VP is initiated using a water-soluble sodium polyacrylate-SG1 macroalkoxyamine. The copolymers obtained assemble to form aggregates, the chain extension taking place on the hydrophobic-chain side. Observations with a transmission electron microscope (TEM) showed that the polymer particles formed are less than a micrometre in size and have variable shapes, ranging from spherical particles (60 nm) to vermiform micelles (60 nm in width), and then to spherical vesicles and elongated vesicles or vesicles with multiple compartments, as shown in FIG. 3 of said document.

The publication by S. Boissé et al. in Chem. Commun. 2010, 46, 1950-1952 presents the preparation of nanofibres constituted of amphiphilic block copolymers by reversible addition fragmentation transfer (RAFT) radical polymerization. Styrene is polymerized in water in the presence of a hydrophilic macromolecular RAFT agent (or RAFT macroagent). When the homopolymer of acrylic acid (AA) or of poly(ethylene glycol) methyl ether acrylate (PEGA) is used as RAFT macroagent, the block polymers obtained are in the form of spherical micelles (as shown in FIG. 1 of the cited article, (1) and (9), respectively).

The publication by Y. Li and S. P. Armes in Angew. Chem. Int. Ed. 2010, 49, 4042-4046 describes the synthesis of a latex of spherical particles of nanometric sizes (20 to 100 nm) by self-assembly of a RAFT-synthesized PGMA-PHPMA diblock copolymer.

The publication by S. Sugihara et al. in Soft Matter, 2011, 7, 10787 describes nanoparticles formed from crosslinked diblock copolymers, said copolymers consisting of PMPC chains and a core of PHPMA crosslinked using EDGMA. Spherical structures are obtained with zero or very low contents of EDGMA.

WO 2010/096 867 describes heterogeneous polymer particles comprising a polymer formed at the surface of another polymer. These particles are obtained by means of a process comprising two polymerization steps:
in a first step, at least one ethylenically unsaturated monomer is polymerized in the presence of a RAFT agent to obtain an aqueous dispersion of grains of polymer particles, which are then crosslinked;
In a second step, an ethylenically unsaturated monomer is polymerized at the surface of said crosslinked particles, to obtain a polymer which will have a structure different from that of said grains.
   The polymers thus obtained are used in aqueous and organic compositions for coatings, cosmetics, adhesives and the like.

None of these documents describes the ageing resistance properties of said spherical particles.

It has now been found, surprisingly, that certain spherical micelles have the property of having increased ageing resistance, especially thermal ageing resistance, in dispersed medium if their cores are chemically crosslinked.

One subject of the invention is the use of a latex of crosslinked spherical polymer particles constituted of block copolymers synthesized by controlled radical emulsion polymerization, for improving the thermal ageing resistance of an aqueous or organic composition. Characteristically, these polymer particles are in the form of spheres with a diameter of from 10 to 100 nm and are obtained by self-assembly of said block copolymers.

According to one embodiment, the synthesis of said particles is performed using at least one hydrophobic monomer and a crosslinking agent in the presence of a living macroinitiator derived from a nitroxide, characterized in that:
said spherical particles are obtained in aqueous medium directly during the synthesis of said block copolymers, performed by heating the reaction medium to a temperature of from 60 to 120° C.,
said macroinitiator is water-soluble,
the percentage of the molar mass of the water-soluble macroinitiator in the final block copolymer is between 10% and 50%, and in that
the degree of conversion of the hydrophobic monomer is at least 50%.

This direct method for preparing crosslinked spherical particles does not require the use of an organic cosolvent.

In the context of the present invention, the term “spherical particles” corresponds to self-assemblies of amphiphilic macromolecules which, when suspended in water (in other words, when they form an aqueous dispersion), spontaneously take the form of a sphere whose core is constituted of the hydrophobic components and whose surface is constituted of the hydrophilic components of said macromolecules. These spherical particles may be observed with a transmission electron microscope (TEM). The microscopy images show spheres whose diameter is greater than or equal to 10 nm.

According to another embodiment, the synthesis of said spherical particles is performed by reversible addition fragmentation transfer (RAFT) radical polymerization in water in the presence of a hydrophilic macromolecular RAFT agent (or RAFT macroagent).

Said aqueous compositions, whose ageing resistance, in particular thermal ageing resistance, is improved, find applications as additives for aqueous or organic solutions.

The invention and the advantages it affords will be better understood in the light of the detailed description that follows and of the attached FIGS. 1 and 2 which illustrate the spherical particles according to the invention viewed by transmission electron microscopy (TEM).

It has now been found that a composition comprising said crosslinked spherical polymer particles has increased ageing resistance, in particular thermal ageing resistance, which allows it to be able to be used for longer and over a wider temperature range than the compositions of the prior art.

Advantageously, chemical crosslinking to the core of the spherical micelle makes it possible to make the latex totally immiscible, even in solvents that dissolve all the polymer. Furthermore, this crosslinking enables the latex of spherical particles in dispersed medium (for example aqueous medium) to not degrade after having undergone thermal ageing for several days at temperatures above 100° C. The same structure obtained by the same self-assembly but which does not benefit from chemical crosslinking of the core will have its structure totally degraded after the same thermal ageing.

To this end, one subject of the invention is the use of a latex of crosslinked spherical polymer particles constituted of block copolymers synthesized by controlled radical emulsion polymerization, for improving the thermal ageing resistance of the composition. Characteristically, these polymer particles are in the form of spheres with a diameter of between 10 and 100 nm. The term “latex” is understood herein to be an aqueous or organic continuous phase in which are dispersed the spherical polymer particles constituted of block copolymers. These particles are constituted of amphiphilic macromolecules which have the capacity of self-assembling.

According to one embodiment, the synthesis of said particles is performed using at least one hydrophobic monomer and a crosslinking agent in the presence of a living macroinitiator derived from a nitroxide.

Characteristically, said crosslinked spherical particles are obtained in the aqueous medium for synthesizing said block copolymers performed by heating the reaction medium to a temperature of 60 to 120° C., with a percentage of the molar mass of the hydrophilic macroinitiator in the final block copolymer of between 10% and 50%, the degree of conversion of the hydrophobic monomer and of crosslinking agent being at least 50%. The crosslinking agent is advantageously introduced into the reaction medium to a content of at least 1% and preferably between 5% and 15% by weight relative to the weight of hydrophobic monomer. The initial pH of the aqueous medium may range between 5 and 10. This direct method for preparing crosslinked spherical particles does not require the use of an organic cosolvent.

The term “living macroinitiator” means a polymer comprising at least one end that is capable of being re-employed in a polymerization reaction by adding monomers at a suitable temperature and pressure. Advantageously, said macroinitiator is prepared by CRP. The term “water-soluble macroinitiator” means a polymer that is soluble in water, comprising at its end a reactive function that is capable of reinitiating a radical polymerization. This macroinitiator is predominantly composed of hydrophilic monomers, i.e. of monomers bearing one or more functions that are capable of establishing hydrogen bonds with water. In case of the polymerization of a hydrophobic monomer, an amphiphilic copolymer will be formed, the hydrophilic block of which will be constituted by the macroinitiator whereas the hydrophobic block will be derived from the polymerization of the hydrophobic monomer(s). According to one embodiment variant, said preformed water-soluble macroinitiator is added to the reaction medium comprising at least one hydrophobic monomer.

According to another variant within the first embodiment, said water-soluble macroinitiator is synthesized in the aqueous reaction medium in a preliminary step, without isolation of the macroinitiator formed or removal of the possible residual hydrophilic monomers. This second variant is a “one-pot” polymerization.

The hydrophobic monomers may be chosen from:
vinylaromatic monomers such as styrene or substituted styrenes,
alkyl, cycloalkyl or aryl acrylates such as methyl, ethyl, butyl, 2-ethylhexyl or phenyl acrylate,
alkyl, cycloalkyl, alkenyl or aryl methacrylates such as methyl, butyl, lauryl, cyclohexyl, allyl, 2-ethylhexyl or phenyl methacrylate,
and vinylpyridine.

These hydrophobic monomers are added to the reaction medium, which predominantly comprises water.

The crosslinking agent used is a crosslinking comonomer other than the abovementioned hydrophobic monomers.

The term “crosslinking comonomer” means a monomer which, by means of its reactivity with the other monomers present in the polymerization medium, is capable of generating a covalent three-dimensional network. From a chemical viewpoint, a crosslinking comonomer generally comprises at least two ethylenic polymerizable functions, which, on reacting, are capable of creating bridges between several polymer chains.

These crosslinking comonomers may be capable of reacting with the unsaturated hydrophobic monomers during the synthesis of said particles.

Among the crosslinking comonomers that may be mentioned are divinylbenzenes, trivinylbenzenes, allyl (meth)acrylates, diallyl maleate polyol (meth)acrylates such as trimethylolpropane tri(meth)acrylates, alkylene glycol di(meth)acrylates containing from 2 to 10 carbon atoms in the carbon chain, such as ethylene glycol di(meth)acrylates, 1,4-butanediol di(meth)acrylates and 1,6-hexanediol di(meth)acrylates, and N,N′-alkylenebisacrylamides, such as N,N′-methylenebisacrylamide. Preferably, divinylbenzene or a dimethacrylate will be used as crosslinking agent.

Use of said process makes it possible to obtain crosslinked spherical polymer particles in which the mass content of the hydrophilic part of which the block copolymer is composed is less than 50%.

Characteristically, the crosslinked spherical particles according to the invention have a molar mass percentage of the hydrophilic macroinitiator in the final block copolymer of between 10% and 50%. Preferably, the molar mass percentage of the water-soluble macroinitiator in the final block copolymer is between 10% and 30%.

As observed by TEM, the crosslinked spherical particles according to the invention are in the form of spheres; their diameter is constant over their entire length and is greater than or equal to 10 nm. The shape and structure of the spherical particles according to the invention is maintained in dispersed medium, independently of their concentration in the medium and/or of the variations in pH or salinity thereof.

According to a second embodiment, the synthesis of said crosslinked spherical particles is performed by reversible addition fragmentation transfer (RAFT) radical polymerization in water in the presence of a hydrophilic macromolecular RAFT agent (or RAFT macroagent).

The structural integrity of the spherical micelles according to the invention is not affected after thermal ageing for several days at temperatures above 100° C. in aqueous medium.

The compositions targeted by the present invention are obtained by adding said crosslinked spherical polymer particles to an aqueous or organic solution to a minimum mass content of 50 ppm. Said compositions, whose ageing resistance, in particular thermal ageing resistance, is improved, are particularly suitable for use as additives in fluids intended for the oil extraction industry (antideposition agent). Other applications of these compositions are directed towards the cosmetic, paints, papermaking and thickener fields.

The invention will now be described with the aid of the following examples, which are given as non-limiting illustrations.

### EXAMPLE 1

### Preparation of the poly(methacrylic acid-co-sodium styrenesulfonate) Macroinitiator In Situ for the Process for Obtaining Spherical Micelles in a Single Step (EG232)

This example illustrates the preparation of a poly(methacrylic acid-co-sodium styrenesulfonate) living copolymer, used as macroinitiator, control agent and stabilizer, as feedstock, for the synthesis of nanoparticles in the form of spherical micelles of poly(methacrylic acid-co-sodium styrenesulfonate)-b-poly(n-butyl methacrylate-co-styrene) block copolymers. The amphiphilic copolymer is synthesized in a single step. The conditions for synthesizing the macroinitiator may be varied (polymerization time, content of sodium styrenesulfonate, concentration and pH) to adapt and vary the composition of the macroinitiator.

To do this, a mixture containing 14.8715 g of methacrylic acid (1.79 mol·L_{aq}⁻¹ or 1.55 mol·L⁻¹), 3.2713 g of sodium styrenesulfonate (1.48×10⁻¹ mol·L_{aq}⁻¹ or 1.28×10⁻¹ mol·L⁻¹, i.e. f_{0.SS}=0.076; f_{0.SS}=n_{SS}/(n_{SS}+n_{MAA})), 0.4101 g of Na₂CO₃ (4.01×10⁻² mol·L_{aq}⁻¹ or 3.48×10⁻² mol·L⁻¹) and 89.1 g of deionized water is degassed at room temperature by sparging with nitrogen for 15 minutes. In parallel, 0.7163 g (1.95×10⁻² mol·L₁⁻¹ or 1.69×10⁻² mol·L⁻¹) of the alkoxyamine BlocBuilder®-MA is dissolved in 7.5394 g of 0.4 M sodium hydroxide (1.6 equivalents relative to the methacrylic acid units of the BlocBuilder®-MA) and degassed for 5 minutes.

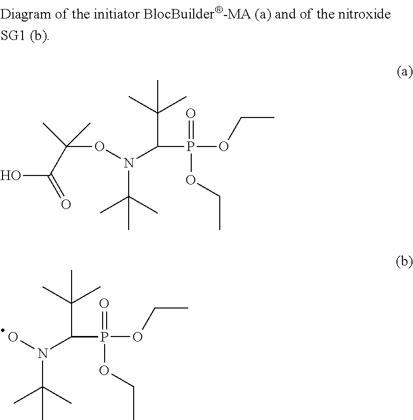 [SMILES: CCOP(=O)(OCC)C(N(OC(C)(C)C(=O)O)C(C)(C)C)C(C)(C)C ; CCOP(=O)(OCC)C(N(O)C(C)(C)C)C(C)(C)C]

The solution of BlocBuilder®-MA is introduced into the reactor at room temperature with stirring at 250 rpm. The monomer solution is then introduced slowly into the reactor. The reactor is subjected to a pressure of 1.1 bar of nitrogen, with continued stirring. Time t=0 is started when the temperature reaches 50° C. The temperature of the reaction medium reaches 65° C. after 15 minutes.

During this reaction, 15.09 g of n-butyl methacrylate and 1.66 g of styrene are placed in a conical flask (solids content=27.2%) and the mixture is degassed by sparging with nitrogen at room temperature for 10 minutes.

After 15 minutes of synthesis of the macroinitiator of poly(methacrylic acid-co-sodium styrenesulfonate)-SG1 type, the t₀ of the emulsion polymerization is started when the second reaction medium containing the hydrophobic monomers is introduced at ambient pressure, and then a pressure of 3 bar of nitrogen with stirring at 250 rpm. The reactor is maintained at 90° C. throughout the polymerization.

Samples are taken at regular intervals in order to determine the polymerization kinetics by gravimetry (solids content measurement).

Table 1 and the attached FIG. 1 show the characteristics of the latices and particles collected from the second step of synthesis of the nanoparticles.

TABLE 1

| Time | Conversion | M_{n, exp}^{a} | Mₙ, theo^{b} | | | Dz^{c} | |
|---|---|---|---|---|---|---|---|
| (h) | (%) | g · mol⁻¹ | g · mol⁻¹ | Iₚ^{a} | pH | (nm) | Σ^{d} |
| 0.03 | 14.63 | — | 5617 | — | — | — | — |
| 0.25 | 61.61 | — | 12854 | — | — | — | — |
| 0.50 | 75.05 | — | 14924 | — | — | — | — |
| 0.75 | 83.66 | — | 16249 | — | — | — | — |
| 1.00 | 87.91 | — | 16904 | — | — | — | — |
| 1.25 | 91.83 | — | 17508 | — | — | — | — |
| 1.50 | 93.76 | — | 17806 | — | — | — | — |
| 1.75 | 94.04 | — | 18019 | — | — | — | — |
| 2.80 | 95.15 | — | 18013 | — | — | — | — |
| 3.30 | 95.10 | — | 18767 | — | 4.41 | 48.9 | 0.177 |

The latex obtained at the end of polymerization is translucent and slightly viscous (high solids content).

Evaluation of the Colloidal Characteristics of the Latex by Dynamic Light Scattering: Mean Particle Diameter, Particle Size Distribution

(a) polydispersity index determined by size exclusion chromatography in DMF with 1 g·L⁻¹ of LiBr, with calibration using polymethyl methacrylate, after methylation of the methacrylic acid units to methyl methacrylate units after purification;

(b) M_{n theo}=M_{n macro}+[(1−0.27)×m_{MAA}+(1−0.54)×m_{SS}+m_{BuMA}+m_{S}]/n_{BlocBuilder®-MA}]×mass conversion with M_{n macro}=M_{BlocBuilder®-MA}+(0.27×m_{MAA}+0.54×m_{SS})/n_{BlocBuilder®-MA}

(c) Mean particle diameter;

(d) Polydispersity of the latex particle sizes.

Measurements (c, d) are taken by dynamic light scattering at 25° C. and in multi-angle calculation mode. The machine is a Zetasizer Nano Series (NanoZS Zen3600) from Malvern Instrument and the software is Zetasizer 6.2. The NanoZS is calibrated with the standard PS latex in water (particle size 200±6 nm). Before the measurements, the samples are diluted with deionized water.

The images are acquired by transmission electron microscopy TEM (ImageJ software). This is a JEOL 100 Cx II microscope at 100 keV equipped with a high-resolution CCD camera, KeenView from SIS.

### EXAMPLE 2

### Preparation of the poly(methacrylic acid-co-sodium styrenesulfonate) Macroinitiator In Situ for the Process for Obtaining Crosslinked Spherical Micelles in a Single Step (EG236)

This example illustrates the preparation of a poly(methacrylic acid-co-sodium styrenesulfonate) living copolymer, used as macroinitiator, control agent and stabilizer, as feedstock, for the synthesis of nanoparticles in the folin of crosslinked spherical micelles of poly(methacrylic acid-co-sodium styrenesulfonate)-b-poly(n-butyl methacrylate-co-styrene) block copolymers. The amphiphilic copolymer is synthesized in a single step.

The conditions for synthesizing the macroinitiator may be varied (polymerization time, content of sodium styrenesulfonate, concentration and pH) to adapt and vary the composition of the macroinitiator.

To do this, a mixture containing 12.1514 g of methacrylic acid (1.51 mol·L_{aq}⁻¹ or 1.34 mol·L⁻¹), 2.6705 g of sodium styrenesulfonate (1.24×10⁻¹ mol·L_{aq}⁻¹ or 1.10×10⁻¹ mol·L⁻¹, i.e. f_{0.SS}=0.076; f_{0.SS}=n_{SS}/(nₛₛ+n_{MAA})), 0.3922 g of Na₂CO₃ (3.95×10⁻² mol·L_{aq}⁻¹ or 3.50×10⁻² mol·L⁻¹) and 87.7 g of deionized water is degassed at room temperature by sparging with nitrogen for 15 minutes. In parallel, 0.5847 g (1.64×10⁻² mol·L_{aq}⁻¹ or 1.45×10⁻² mol·L⁻¹) of the alkoxyamine BlocBuilder®-MA is dissolved in 6.1513 g of 0.4 M sodium hydroxide (1.6 equivalents relative to the methacrylic acid units of the BlocBuilder®-MA) and degassed for 5 minutes.

The solution of BlocBuilder®-MA is introduced into the reactor at room temperature with stirring at 250 rpm. The monomer solution is then introduced slowly into the reactor. The reactor is subjected to a pressure of 1.1 bar of nitrogen, with continued stirring. Time t=0 is started when the temperature reaches 50° C. The temperature of the reaction medium reaches 65° C. after 15 minutes.

During this reaction, 12.00 g of n-butyl methacrylate and 1.35 g of styrene are placed in a conical flask (solids content=23.8%) and the mixture is degassed by sparging with nitrogen at room temperature for 10 minutes.

After 15 minutes of synthesis of the macroinitiator of poly(methacrylic acid-co-sodium styrenesulfonate)-SG1 type, the t₀ of the emulsion polymerization is started when the second reaction medium containing the hydrophobic monomers is introduced at ambient pressure. A pressure of 3 bar of nitrogen and stirring at 250 rpm are applied. The reactor is maintained at 90° C. throughout the polymerization.

After 45 minutes of polymerization of the hydrophobic monomers, 0.412 g of ethylene glycol dimethacrylate (f_{0,EGDMA}=0.021 mol) f_{0,EGMDA}/(n_{EGMDA}+n_{MABu}+n_{Sty}) (solids content=24.0%) is introduced into the reactor to crosslink the spheres after their formation.

In parallel with the reaction, 0.051 g of HSA (sodium formaldehydesulfoxylate) is dissolved in 1.9497 g of deionized water, and 0.0454 g of tBHP (tert-butyl hydroperoxide, 70 wt % H₂O) is diluted in 1.9569 g of deionized water (f_{0,tBHP}=0.0035 mol) (f_{0,tBHP}=n_{tBHP}/(n_{tBHP}+n_{EGDMA}+n_{MABu}+n_{Sty}), i.e. n_{tBHP}/n_{HSA}=1.07). At 172 minutes, the temperature of the reaction medium is set at 70° C. and the solution of HSA is introduced, followed by adding 1 mL of deionized water to rinse the pipe. At 183 minutes, the solution of tBHP is introduced, followed by adding 1 mL of deionized water to rinse the pipe. Cooking is performed for 1 hour 30 minutes.

Samples are taken at regular intervals in order to:

determine the polymerization kinetics by gravimetry (solids content measurement);

evaluate the colloidal characteristics of the latex by dynamic light scattering: mean particle diameter, particle size distribution (polydispersity).

Table 2 below and the attached FIG. 2 show the characteristics of the latices and particles collected from the second step of synthesis of the nanoparticles.

TABLE 2

| Time | Conversion | M_{n, exp}^{a} | Mₙ, theo^{b} | | | Dz^{c} | |
|---|---|---|---|---|---|---|---|
| (h) | (%) | g · mol⁻¹ | g · mol⁻¹ | Iₚ^{a} | pH | (nm) | Σ^{d} |
| 0.25 | 0.4777 | — | 3365 | — | — | — | — |
| 0.72 | 0.7669 | — | 10625 | — | — | 33.23 | 0.174 |
| 1.25 | 0.8685 | — | 15021 | — | — | — | — |
| 1.75 | 0.8848 | — | 16565 | — | — | — | — |
| 2.32 | 0.8919 | — | 16812 | — | — | — | — |
| 2.75 | 0.8941 | — | 16920 | — | — | — | — |
| 4.55 | 0.9127 | — | 16953 | — | 4.69 | 49.07 | 0.263 |

(b) M_{n theo}=M_{n macro}+[(1−0.27)×m_{MAA}+(1×0.54)×m_{SS}+m_{BuMA}+m_{S}]/n_{BlocBuilder®-MA}]×mass conversion with M_{n macro}=M_{BlocBuilder®-MA}+(0.27×m_{AA}+0.54×m_{SS})/n_{BlocBuilder®-MA}

(c) Mean particle diameter;

(d) Polydispersity of the latex particle sizes.

The latex obtained at the end of polymerization is translucent and very slightly viscous.

Evaluation of the colloidal characteristics of the latex by dynamic light scattering: mean particle diameter, particle size distribution

The crosslinking agent is added at 77% of conversion to allow self-assembly of the spherical particles and to avoid the absence of coagulum when too much EGDMA is added.

Abbreviations:

CRP—controlled radical polymerization

P4VP—poly(4-vinylpyridine)

PNaA—poly(sodium acrylate)

SG1—N-tert-butyl-N-[1-diethylphosphono-(2,2-dimethylpropyl)]

S or Sty—styrene

SS—sodium styrenesulfonate

AA—acrylic acid

PEGA—poly(ethylene glycol) methyl ether acrylate

PGMA—poly(glyceryl monomethacrylate)

PHPMA—poly(hydroxypropyl methacrylate)

PMPC—poly(2-methacryloyloxyethylphosphorylcholine)

EGDMA—ethylene glycol dimethacrylate

TEM—transmission electron microscopy

RAFT—reversible addition fragmentation chain transfer

MAA—methacrylic acid

DMSO—dimethyl sulfoxide

DMF—dimethylformamide

rpm—rotations per minute

f_{0,sty}—initial mole fraction of styrene sodium sulfonate in the monomer mixture

f_{0,SS}—initial mole fraction of styrene sodium sulfonate in the monomer mixture

f_{0,DVP}—initial mole fraction of divinylbenzene in the monomer mixture

BlocBuilder®-MA—(N-(2-methylpropyl)-N-(1-diethylphosphono-2,2-dimethylpropyl)-O-(2-carboxylprop-2-yl)hydroxylamine

1. A method for improving the thermal ageing resistance of an aqueous or organic composition, comprising using a latex of crosslinked spherical polymer particles wherein said particles have a diameter of from 10 to 100 nm and are constituted of block copolymers synthesized by controlled radical emulsion polymerization, said latex being obtained by self-assembly of said block copolymers.

2. The method according to claim 1, wherein said particles are synthesized using at least one hydrophobic monomer and a crosslinking agent in the presence of a living macroinitiator derived from a nitroxide, under the following conditions:
said crosslinked spherical particles are obtained in aqueous medium during the synthesis of said block copolymers, performed by heating the reaction medium to a temperature of from 60 to 120° C.,
said macroinitiator is water-soluble,
the molar mass percentage of the water-soluble macroinitiator in the final block copolymer is between 10% and 50%, and in that
the degree of conversion of the hydrophobic monomer is at least 50%.

3. The method according to Claim 1, wherein the hydrophobic monomer is selected from the group consisting of vinylaromatic monomers, alkyl, cycloalkyl and aryl acrylates, alkyl, cycloalkyl, alkenyl and aryl methacrylates, and vinylpyridine.

4. The method according to claim 1, wherein the molar mass percentage of the water-soluble macroinitiator in the final block copolymer is between 10% and 30%.

5. The method according to claim 1, wherein the mass content of the hydrophilic part of which the final block copolymer is composed is less than 50%.

6. The method according to claim 1, wherein said crosslinking comonomer is selected from the group consisting of divinylbenzenes, trivinylbenzenes, allyl (meth)acrylates, diallyl maleate polyol (meth)acrylates and alkylene glycol di(meth)acrylates containing from 2 to 10 carbon atoms in the carbon chain.

7. The method according to claim 1, wherein the crosslinking agent is introduced into the reaction medium to a content of at least 1% by weight relative to the weight of hydrophobic monomer.

8. The method according to claim 1, wherein said composition is obtained by adding said spherical polymer particles to an aqueous or organic solution to a minimum mass content of 50 ppm.

9. The method according to claim 8, wherein said composition is used as an antideposition agent during oil extraction.

10. The method according to claim 1, wherein said composition is used for preparing paints.

11. The method according to claim 1, wherein said composition is a cosmetic composition.
